(19) United States
(12) Patent Application Publication
(10) Pub. No.: US 2017/0216615 A1
(43) Pub. Date: Aug. 3, 2017

(54) PLASMA TREATMENT OF AN INFECTED NAIL OR INFECTED SKIN

(71) Applicant: LINDE AKTIENGESELLSCHAFT, Munich (DE)

(72) Inventors: Nick Christopher Pledge, Windsor (GB); Thomas Bickford Holbeche, Church Crookham (GB)

(21) Appl. No.: 15/500,932

(22) PCT Filed: Aug. 4, 2015
(86) PCT No.: PCT/EP2015/067997

§ 371 Date: Feb. 1, 2017

(30) Foreign Application Priority Data
Aug. 5, 2014 (GB) 1413826.7

(51) Int. Cl. A61N 1/44; A61L 2/00

(52) U.S. Cl. CPC A61N 1/44; A61L 2/0011; A61L 2202/11

(57) ABSTRACT

A non-thermal plasma for use in the treatment of an infected nail or infected skin. The plasma is used in the method by
(a) applying the plasma to the infected nail or skin;
(b) rehydrating the infected nail or skin;
(c) applying the plasma to the infected nail or skin; and
(d) optionally rehydrating the infected nail or skin.

In each of steps (a) and (c) the plasma is applied to a portion of the nail or skin until a hydration level of the plasma-treated portion drops by at most 30 wt % based on the initial moisture content of the plasma-treated portion.

The present invention relates to the treatment of an infected nail or infected skin with plasma. The present disclosure relates in particular to a non-thermal plasma treatment using a so-called “cold plasma”. The treatments are preferably carried out in a medical or professional environment, or in the comfort of a user's home environment.

A gas is normally an electric insulator. However when sufficient thermal energy is supplied to a gas or, alternatively, a sufficiently large potential difference is applied across a gap containing a gas, then it will breakdown and conduct electricity. This is because the electrically neutral atoms or molecules of the gas have been ionised to form electrons and positively charged ions. This ionised gas is a plasma.

When the ionisation is driven by a large potential difference, the momentum transfer between the light electrons and the heavier gas molecules and plasma ions is not very efficient. Therefore, the bulk of the energy that is supplied to form the plasma is supplied to the electrons. As a result ionised gases, particularly at low gas pressures and charged particle densities, are described as “cold” or non-thermal. This means that the constituents e.g. the electrons, ions and gas molecules are each in thermal equilibrium only with similar mass species.

Such non-thermal plasmas are well known for use in destroying bacteria. For this reason, it is known to use non-thermal plasma in various forms of dental surgery. Due to the restrictions when operating in a patient's mouth, such plasma devices typically rely on a flow of gas between two electrodes to produce the plasma which can be directed onto the treatment area. The non-thermal production of the plasma provides a plasma gas having a temperature which is tolerable for the patient. WO2013040476 discloses a dental treatment device.

It is also known to use plasma devices for treating skin infections. In such applications, the skin of the patient is typically used to provide the second electrode. In this way a first electrode can be held over the area to be treated and a large voltage difference is formed between the electrode and the patient's skin. This leads to the formation of a plasma from the gas between the electrode and the patient's skin. This allows for treatments of large areas through the use of a large electrode, but relies upon the formation of plasma from the air layer. U.S. Pat. No. 8,103,340, for example, uses such a device for treating a patient's skin.

It is known that the nature of the breakdown and the voltage at which this occurs varies with a wide number of parameters including the gas, the gas pressure, the materials and the nature, geometry and separation of the surfaces across which the potential difference is sustained, the separation distance of the electrodes and the nature of the high voltage supply.

It is known to apply a sensitising agent to the surface of a treatment area when seeking to treat diseases such as onychomycosis. For example, as disclosed in WO2013040542 a sensitising agent may be sprayed onto a surface of a nail during treatments to wet the surface and to interact with a plasma gas on the treatment site. The plasma may be applied before and after the wetting has been performed. The sensitising agents may be water.

WO2013/140371 relates to a method for processing keratinous fibers, such as hair for the production of wigs. The method involves plasma bleaching of the fibers for 1 second to 60 minutes.

WO2014/143412 relates to a method and apparatus for antimicrobial treatment, such as for treating onychomycosis. The method involves the application of non-thermal plasma in combination with ultrasound and is said to provide markedly better cure rates.

WO2010/072997 relates to a method for the cosmetic whitening of teeth in vivo. The method involves the provision of a flow of plasma gas to a tooth at an acceptable temperature for the treatment. In the exemplary treatments the teeth, obtained from a freshly sacrificed pig's jaw, were kept moistened to simulate a realistic dental environment.

GB2501484 relates to a tooth treatment device. The device relies on the provision of non-thermal plasma to a treatment site and incorporates an electrode at the plasma outlet to reduce the number of electrons in the plasma gas leaving the device.

It is an object of the present invention to provide an improved approach to the use of plasma for various treatments, tackle the drawbacks associated with the prior art, or at least provide a commercially viable alternative thereto.

Accordingly, in a first aspect the present invention provides non-thermal plasma for use in the treatment of an infected nail or infected skin, wherein the plasma is used in a method comprising:

(a) applying the plasma to the infected nail or skin;

(b) rehydrating the infected nail or skin;

(c) applying the plasma to the infected nail or skin; and

(d) optionally rehydrating the infected nail or skin.

wherein the plasma is applied to a portion of the nail or skin in each of steps (a) and (c) until a hydration level of the plasma-treated portion drops by at most 30 wt % based on the initial moisture content of the plasma-treated portion, preferably by at most 20 wt %.

The present disclosure will now be described further. In the following passages different aspects/embodiments of the disclosure are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

The present invention relates to the treatment of an infected nail or infected skin. As will be appreciated, depending on the type of plasma generation device selected, the treatment may be applied to an entire nail or a portion of the nail. The treatment may be applied to an entire infected skin region or to only a portion. As a result, when treating only a portion of a nail or skin region it may be necessary to carry out a number of sequential plasma treatments. These may be carried out across the infected region in steps (a) and (c). The rehydration in steps (b) and (d) can more readily be applied to the entire treated area and may each preferably be carried out in a single complete treatment.

As discussed above, the plasma used in the present method is a cold or “non-thermal” plasma. This is essential when treating the human body since a thermal plasma would cause very severe tissue damage.

The method of the invention comprises sequentially and repeatedly applying the plasma to the infected nail or skin and rehydrating the plasma-treated nail or skin. In this way the hydration of the treated area can be maintained. It has been found that this results in a faster and more effective treatment and it is theorised that this may be due to the presence of water-derived radicals which may be formed within the skin or nail. It is particularly surprising that the rehydration is so effective since it is known that the presence of high moisture levels in the air can hinder the production and longevity of the plasma.

As noted above, WO2013040542 discloses that water may be sprayed with a plasma gas onto the surface of a treatment site. However, the present inventors have found that such a method hinders plasma stability which is required to provide the necessary chemical species in the nail for efficacious treatment. In addition, the method of WO2013040542 results in water on the surface to be treated which acts as a barrier which prevents passage of active compounds into the nail to effect treatment. Moreover, brief surface wetting by applying water to a nail surface does not achieve proper rehydration of the nail back to substantially its original hydration. Indeed, this has been found to be best achieved by immersing the nail in water for at least 5 seconds (depending on the amount of dehydration).

The present invention involves the penetration of treatment into the nail or skin by maintaining the hydration of the nail or skin throughout the depth to be treated. By portion, therefore, it is intended to refer not just to the surface but also to the full thickness of a nail or infected skin region. When treating an infected nail the fungus will often be present in the nail bed and it is critical to treat this area: maintaining hydration throughout the nail has been found to facilitate this treatment.

In particular, the inventors have found that the use of plasma allows for an efficacious treatment of infections of the nail and skin. The effect of the plasma has been found to effectively penetrate nails to treat the nail bed and can destroy an infection over a much shorter treatment time than a conventional cream treatment. However, the inventors have found that, surprisingly, the efficacy of the treatment can be significantly increased if the hydration of the treated portion can be maintained.

Without wishing to be bound by theory, the inventors speculate that the plasma treatment of an infected nail or skin region is driven by a number of mechanisms fuelled by the production of plasma-derived reactive oxygen and nitrogen species (RONS). These may be formed in part with the moisture in the nail and, therefore, it is critical that the nail moisture is maintained.

In particular, it is proposed that plasma treatment exerts its fungicidal action through the disruption of the cell exterior by increasing its permeability, resulting in a loss of membrane integrity and leakage of intracellular components. This cell death by necrosis may be mediated through more than one mechanism:
Production of transient pores by lipid and polysaccharide peroxidation induced by plasma-derived reactive oxygen and nitrogen species (RONS);
Oxidation by RONS of protein thiol groups in the cell membrane and wall leading to their degradation;
Electroporation due to the residual electric current released from the device if the electric field exceeds ˜50 kV/cm.

It is hypothesized that the RONS generated from the interaction of ionised gas jet with air interact with water in nail, eventually creating OHONO (peroxynitrous acid). This molecule would act as an intermediate agent that permeates through the nail releasing OH which is most likely the final active species acting on the fungal cell.

Programmed cell death or apoptosis, another recognised cellular effect of plasma treatment in general, is believed to be a less relevant fungicide mode of action, except perhaps in the case of fungal spores. Apoptosis can occur when a compromised membrane structure (e.g. peroxidation) or change in membrane-bound proteins (e.g. ion channel proteins) activates intracellular signal pathways leading to complex cell responses ending in apoptosis. On the other hand, plasma-generated RONS themselves may penetrate into the cytoplasm inactivating the functional enzymes and other components within the cell, and inducing direct damage of DNA resulting in apoptosis.

UV radiation is likely to have a modest role in fungicidal action. Heat is not considered relevant in the efficacy of plasma as the induced surface temperature is below that resulting in thermal cell damage.

However, the application of plasma to a surface of a nail or skin may have an undesirable drying effect. This is because, despite the term “non-thermal”, the plasma is associated with a greater-than ambient temperature and prolonged application can lead to a thermal build-up. In addition, the flow of plasma acts like a flow of gas conducting moisture away from a surface across which it is directed. Accordingly, prolonged plasma treatment can have a desiccating effect.

The inventors have also found that the extent of dehydration of the area treated is also important. The amount of moisture lost from a nail or skin patch is related to the time required for rehydration. In tests it was found that a completely dehydrated nail could not be rehydrated except after two hours of immersion in water, whereas if the moisture loss was less than 30 wt % of the original nail moisture, then this could be restored within less than 5 minutes of rehydration.

Preferably the method comprises repeating steps (b) and (c) at least once, preferably at least 3 times, preferably from 5 to 15 times and more preferably about 10 times. An advantage of performing the method repeatedly with sequential and repeated treatments is that the hydration level of the treated area is not too significantly reduced and may be quickly restored.

Preferably the steps (a)-(d) do not overlap. This avoids the presence of water suppressing the production of the plasma required for the treatment. Similarly, it is desirable for the treated surface to be kept clear of excess water, by wiping any surface water from the area to be treated or drying it therefrom. This avoids the presence of water acting as a barrier to the actives produced entering the skin or nail.

The plasma-treatment method is effective in the treatment of nail or skin infected with a viral or fungal infection. In particular, the plasma-treatment method can be used to treat nail or skin infected with Trichophyton rubrum, which provides nail fungus (Onychomycosis) and athletes foot, or skin infected with a human papillomavirus which causes wart (also known as verrucas). The use of the plasma-treatment method serves to ameliorate the infection and to reduce the risk of the disease spreading or reoccurring.

Onychomycosis is a nail disease of the toes and fingers typically caused by the organisms Candida albicans, Trichophyton mentagrophytes, Trichophyton rubrum, or Epidermpophyton floccusum. The nails become thickened and lustreless, and debris accumulates under the free edge. Nail plates becomes separated and the nails may be destroyed. It is acknowledged that the therapy of onychomycosis is difficult and protracted. Oral therapy with antimycotics requires months of administration and must be closely monitored for side effects.

Topical compositions have long been used with the objective of treating onychomycosis. Yet these chemical based topical applications have been largely unsuccessful because the nail is a difficult barrier for anti-fungal compounds to penetrate. To be effective a topical treatment for onychomycosis should exhibit a powerful potency for pathogens. It must also be permeable through the nail barrier, and safe for patient use. There exists a need in the art for a topical application that combines these traits in high degree.

The compositions and method of the invention provide a unique means for treating these infections such as onychomycosis. Advantageously, such means provides, in combination, certain characteristics, including safety, effectiveness, convenience, and freedom from toxicity, which have been unavailable heretofore. Through in vitro microbiological tests it is now found that a topical application of Plasma using the method described herein, a topical application regime can be provided to a patient to effectively penetrate the nail and kill off the fungus causing the disease.

Preferably the plasma is provided by an electrical discharge through a gas, preferably through a gas selected from Helium, Oxygen, Argon, Krypton, Neon, Air, Hydrogen, and mixtures of two or more thereof. In particular, gases such as Helium, Argon, Krypton and Neon are known to form long lasting stable ions which can lead to a stabilised plasma. The use of air is convenient and, indeed, may avoid the need for gas storage. However, the presence of large amounts of oxygen can lead to the production of undesirable ozone levels. Accordingly, except when working in air, it is preferred that the oxygen is present in an amount of less than 1000 ppm, more preferably less than 500 ppm. Hydrogen has a high thermal conductance and can be used to avoid the build-up of heat, but it can quench the production of plasma; preferably it is present in an amount of from 1 to 4 v %.

Preferably in each of steps (a) and (c) the plasma is applied to a portion of the nail or skin to be treated for a period of from 5 seconds to 2 minutes, more preferably from 15 second to 90 seconds and most preferably from about 30 seconds to about 1 minute. Accordingly, if the treatment area of the plasma device is quite small, it will need to be moved across an infected area to treat it fully. In this case, preferably each area treated is treated for the above mentioned period. Thus, for example, if a nail is sized in comparison to the plasma treatment device such that it requires eight separate portions to be treated, a total treatment time of 4 minutes would allow 30 seconds of treatment for each portion.

Preferably the skin or nail is rehydrated in each of steps (b) and (d) by applying water to the skin or nail. This is the simplest manner and can be achieved using a damp cloth or a receptacle of water for immersion. Alternatively rehydration can be achieved using high humidity air or a hydrogel.

Preferably after the skin or nail is rehydrated in each of steps (b) and (d) by applying water to the nail, excess water is removed from the skin or nail. It is, as noted above, not desirable to have free water on a surface to be plasma treated.

Preferably water is applied to the skin or nail for a period of from 15 seconds to 6 minutes. More preferably the water is applied for a period of from 1 minute to 3 minutes and most preferably about 2 minutes.

Preferably the plasma is applied to the nail or skin in each of steps (a) and (c) until a hydration level of a plasma-treated portion drops by at most 30 wt % based on the initial moisture content of the nail or skin, preferably by at most 20 wt %. The moisture content of a nail or skin can be determined using Raman spectroscopy. For efficiency of the procedure it is preferred that the moisture drop is at least 1 wt %, more preferably from 2 to 10 wt %, more preferably from 3 to 5 wt %, based on the initial moisture content of the nail (skin).

The moisture content of a nail is typically from 7 to 25 wt % by weight of a nail in vivo. More commonly the moisture is from 10 to 20 wt % and this will depend on the health, condition and environment of a nail, The treatment is preferably designed such that the moisture content of a nail can be predicted and undue drying can be avoided. As will be appreciated, if a nail has an initial moisture content of 15 wt %, then preferably this will not drop to less than 10.5 wt % (a 30 wt % drop in moisture).

Preferably rehydrating the nail or skin in each of steps (b) and (d) restores a hydration level of the nail or skin substantially to the initial moisture content of the untreated nail or skin. That is, preferably the hydration level is restored to at least 90% of the original level, more preferably at least 95% and most preferably at least 97%.

Preferably the method comprises:

(A) applying the plasma to the nail for a period of about 30 seconds;

(B) rehydrating the nail by applying water to the nail for a period of about 2 minutes; and

(C) repeating steps (A) and (B) at least three times,

This treatment regime has been found to be effective and to avoid undue drying of the nail. Full rehydration can be achieved in the time available.

According to a second aspect there is provided a non-therapeutic method for lightening the colour of a nail, wherein the method comprises:

(a) applying non-thermal plasma to at least a portion of the nail;

(b) rehydrating said portion the nail;

(c) applying non-thermal plasma to said portion of the nail; and

(d) optionally rehydrating said portion of the nail.

The lightening treatment serves to remove discoloration that may be present in particular when a nail is infected, such as with a fungal infection.

Preferably, before step (a), the infected nail or skin is filed or abraded to pre-prepare the surface to be treated. This is particularly the case for nails where the removal of any surface covering, introducing surface roughness and reducing the thickness to be treated, has been found to increase the efficacy of the treatment and to reduce rehydration times. Without wishing to be bound by theory it is considered that the filing reduces the bather to reintroducing water into the nail.

The inventors have discovered that the treatment results in effective bleaching a treated nail. It is typically the case that an infected nail will show some discolouration and will be yellowed. It is also known that the nails of smokers can become discolored and even painted nails can retain some unwanted colouration when the paint is removed. The inventors have found that the method can quickly to reduce the coloration of such nails so that they are lightened and a more natural colouration can be recovered.

The present invention relies on the use of a plasma-generation device. That is, a device designed to produce a plasma from the ionisation of a gas. The device is especially for producing a non-thermal plasma, as discussed herein. The plasma produced preferably has a temperature of less than 50° C., more preferably less than 47° C. and most preferably from 37 to 45° C., more preferably from 39 to 42° C. The device is suitable for applying plasma to a human body, which applies a number of constraints since thermal plasma production devices are clearly unsuitable. Furthermore, the production levels of UV, electrical stimulation and active species must be at levels which do not cause undue harm to a patient.

The device described herein is preferably hand-held. By hand-held, it is meant that at least the treatment application head is sized and configured such that it can be readily manipulated and controlled with one hand. The treatment application head may be tethered to a power supply and/or a gas reservoir. Alternatively the treatment head may be fixed or pivotable with relation to an area to be treated. The device may also, for example, take a form such as a foot spa to allow ready treatment of an infected foot.

The ideal form for home use by a consumer is an entirely self-contained hand held device. This would have an internal battery as a power source and rely upon interchangeable gas canisters which can be clipped into the device. Nonetheless, for reasons of power requirements, it may be easier to have a mains power lead attached to the device.

Especially when the device is to be used by a professional, such as in a nail salon, or by a doctor, podiatrist, or the like, it may be easier to have the hand-held device tethered to a power supply and a larger gas tank. This makes it easier for the professional to use since they do not need to change the gas tank/cartridge/canister often,

Preferably the power supply comprises a battery integrated into a hand-held device. That is, preferably the plasma-generation device is entirely independent and does not require a tether to a power supply. This increases the utility of the device in-so-far as it can be more accurately applied and can be used in a wider range of environments, such as bathrooms,

The use of the device as discussed herein has a large number of advantages. The provision of the plasma keeps the device sterile and it can be readily reused for multiple patients. In addition, the plasma produces a ready supply of active gas species which provide the treatments discussed herein. The active gas species are further supplemented by the temperature, UV light and electrical stimulation which are associated with the plasma production process.

The plasma treatment device comprises a reservoir containing the above-discussed gas. The reservoir acts as a source of gas from which a plasma is generated. The reservoir contains a source of pressurised gas which can be supplied to the plasma zone as the treatment application portion of the device. The gas will typically be stored in a tank (up to approximately 200 L) for professional use, or in replaceable and/or rechargeable canisters of cartridges for home use. The use, design and requirements for such sources of gas are well known in the art.

The reservoir is in fluid communication with a plasma zone within which plasma is created for treatment. In some embodiments the plasma zone is within the device and a flow of the plasma which is created leaves the device to provide the treatment. In other embodiments the plasma is formed directly at the site to be treated. The plasma zone includes means for generating a plasma by electrical discharge therein.

The device comprises a means for generating a plasma by electrical discharge through the gas. This can be achieved by one of several different approaches.

According to a first approach, using a so-called dielectric barrier discharge, the means for generating a plasma comprises a power supply and a dielectric electrode for placing in proximity to a human body, and wherein, in use, the plasma zone is formed between the dielectric electrode and a surface of a human body. The provision of a high voltage drop between the dielectric electrode and the human body leads to the production of a plasma between the dielectric electrode and the body. This is an effective way to treat a large area. The device of the present invention would preferably be configured such that the gases discussed herein can be flowed into the space formed between the dielectric electrode and the body, preferably at a relatively low flow rate, across substantially the whole area of the electrode.

According to a second approach, the means for generating a plasma is a so-called surface micro discharge device. This comprises a power supply and first and second electrodes sandwiching a dielectric material. In use, a plasma zone is formed adjacent a surface electrode which can be held close to a surface of a human body. The provision of a high voltage drop between the electrodes leads to the production of a plasma across the area and, indeed, the electrode close to the treatment area will typically be a wire mesh. This is an effective way to treat a large area. The device of the present invention would preferably be configured such that the gases discussed herein can be flowed into the space formed between an external electrode on the device and the body, preferably at a relatively low flow rate, across substantially the whole area of the electrode.

According to a third approach the means for generating a plasma is a so-called jet device, and comprises a power supply, and first and second electrodes, and wherein, in use, the plasma zone is formed between the first and second electrodes and wherein a flow of gas from the reservoir through the plasma zone provides a flow of plasma to contact a surface of a human body. The provision of a high voltage drop between the two electrodes will cause the production of a plasma by ionising the gas provided. In this embodiment the gas flow will typically be greater so that the plasma flows out from between the electrodes and can be applied to a treatment area.

Preferably the means for generating a plasma operates at a voltage of from 2-15 kV, preferably from 3 to 10 kV and most preferably about 5 kV. These levels of voltage can be achieved in a hand-held device and still produce a suitable level of plasma generation. The power range of the device is preferably 1-100 Watts AC at a high frequency of 10-60 KHz. Alternatively, power may be delivered as high frequency pulsed DC fast rise time square waveforms.

Preferably the gas is supplied through the means for generating a plasma at a flow rate of less than 1.5 l/min, preferably from 0.01 to 0.5 l/min. The gas flow rate for area treatments as discussed above will typically be lower than required for point treatments which require the production of a targeted jet of plasma. The flow rates for treatments which produce a plasma between a dielectric electrode a treatment are of a patient are preferably from 0.01 to 0.1 l/min. The flow rates for treatments which produce a plasma between two electrodes and rely on the gas flow to carry the plasma to a treatment are preferably from 0.2 to 0.5 l/min,

A preferred device for the treatment of nails is a foot-spa. Such a device would be designed to provide one or more plumes of plasma for treating a user's nails.

According to a further aspect, the present invention provides non-thermal plasma for use in the treatment of an infected nail or infected skin, wherein the plasma is used in a method comprising:

(a) applying the plasma to the infected nail or skin;

(b) rehydrating the infected nail or skin;

(c) applying the plasma to the infected nail or skin; and

(d) optionally rehydrating the infected nail or skin

wherein the skin or nail is rehydrated in each of steps (b) and (d) by applying water or a water-containing composition to the skin or nail and wherein, before step (c), excess water is removed from the skin or nail.

According to a further aspect, the present invention provides a method for treating an infected nail or infected skin, comprising the use of non-thermal plasma, the method comprising:

(a) applying the plasma to the infected nail or skin;

(b) rehydrating the infected nail or skin;

(c) applying the plasma to the infected nail or skin; and

(d) optionally rehydrating the infected nail or skin.

wherein the plasma is applied to a portion of the nail or skin in each of steps (a) and (c) until a hydration level of the plasma-treated portion drops by at most 30 wt % based on the initial moisture content of the plasma-treated portion, preferably by at most 20 wt %.

All embodiments and features disclosed in relation to the non-thermal plasma for use described herein apply equally to the method of this aspect.

The invention will now be described further in relation to the following non-limiting examples.

Nail Treatment

In view of the apparent fungal kill through nails through a hydrated active intermediate, we needed to determine how much dehydration of the nail was taking place during treatment, and then how quickly this could be recovered by treating the nail with liquid water.

Nails were first weighed and exposed to a standard plasma treatment (1% Ar/He, 2.5 l/min, T=39 C) for 1, 5 and 15 minutes, after which they were reweighed to determine the degree of dehydration. An alternative treatment (4% Kr/Ar, 2.5 l/min, T=42 C) was also conducted, but just for 1 minute.

The nails were placed in a drop of deionised (˜100 μl) water for 1, 5 and 15 minutes. With such small nail samples this is easier than putting water on the nail. It is also simulating having the inner surface constantly being rehydrated from water saturated air. After this time the surfaces were blotted dry and reweighed. This was repeated at 1, 5, and 15-minute intervals to produce a rehydration curve.

In the case of filed nails, they were filed prior to the initial weighing. Filing was done using a standard nail file and removing sufficient material to remove the “shiny” surface when examined with an eyeglass.

Tests with 1% Ar/He mix carried out at 14 kHz/5% Duty/2.5 SLPM. N samples for 1 min, 1 min filed and 5 min treatments, with 9 samples per condition. For 15 mins treatment, this was reduced to 2 samples.

Data are plotted as the percentage of weight recovered with time, so if the nail becomes fully rehydrated it would reach 100%. Data are shown with standard deviation bars.

| | | Time of Exposure/ | |
|---|---|---|---|
| Gas Mixture | Device | min | % Weight loss |
| 1% Ar/He | PF4v2 | 1 | 10 |
| 1% Ar/He | PF4v2 | 1 | 13 |
| (filed) | | | |
| 1% Ar/He | PF4v2 | 5 | 24 |
| 1% Ar/He | PF4v2 | 15 | 31 |
| 4% Kr/Ar | PF4v1 | 1 | 9 |

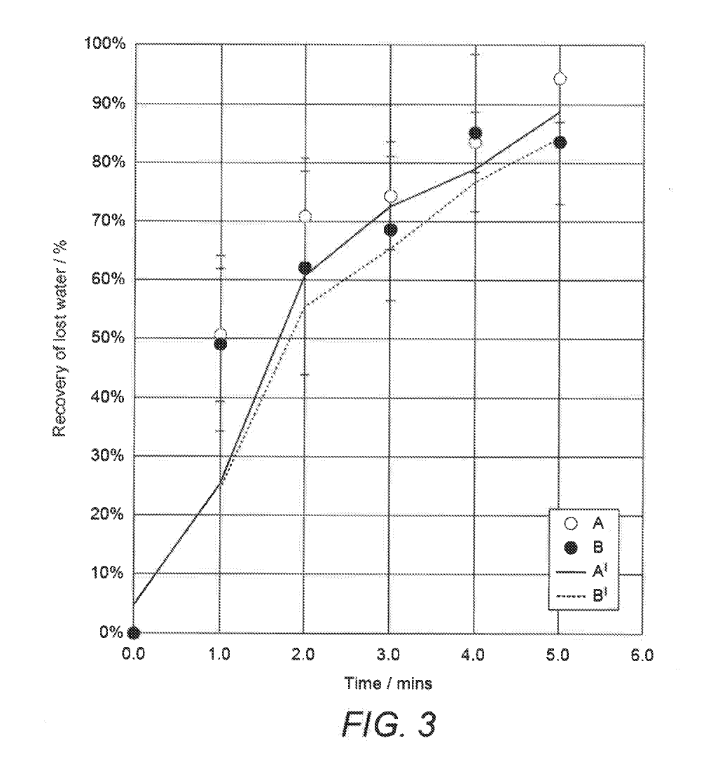

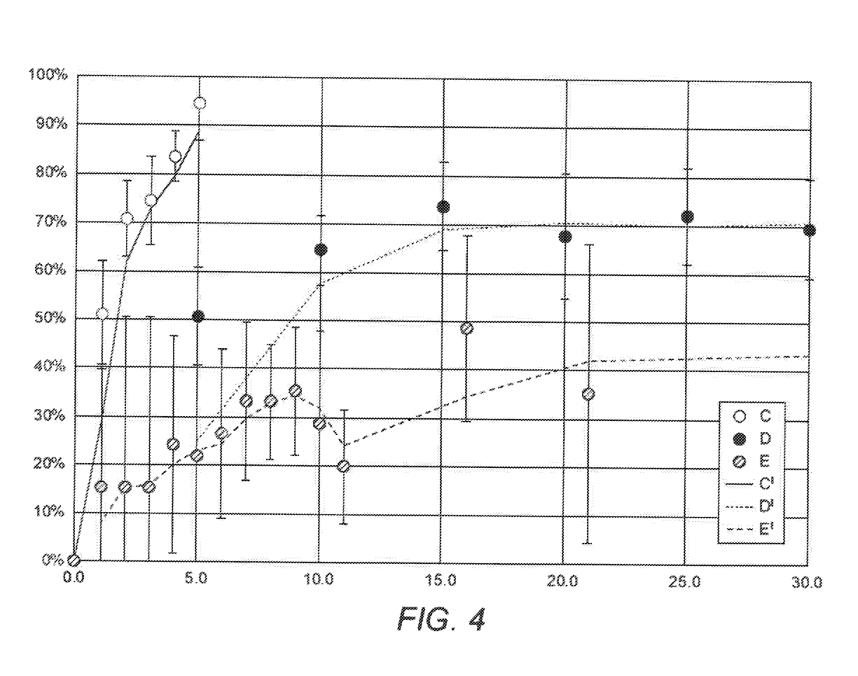

Based on these examples it was found that the longer the treatment the longer it takes to rehydrate, as shown in FIGS. 3 and 4.

The longer the treatment the worse the overall level of rehydration.

Filing the nail seems to increase the overall loss of water during treatment and has a slight effect on the rate of rehydration.

There appears little difference in the degree and rate of rehydration between two different gas treatments based on the same flow speed.

For a better understanding of the invention and to show how the same may be put into effect, preferred embodiments of the invention will now be described, by way of example only, with reference to the accompanying drawings in which:

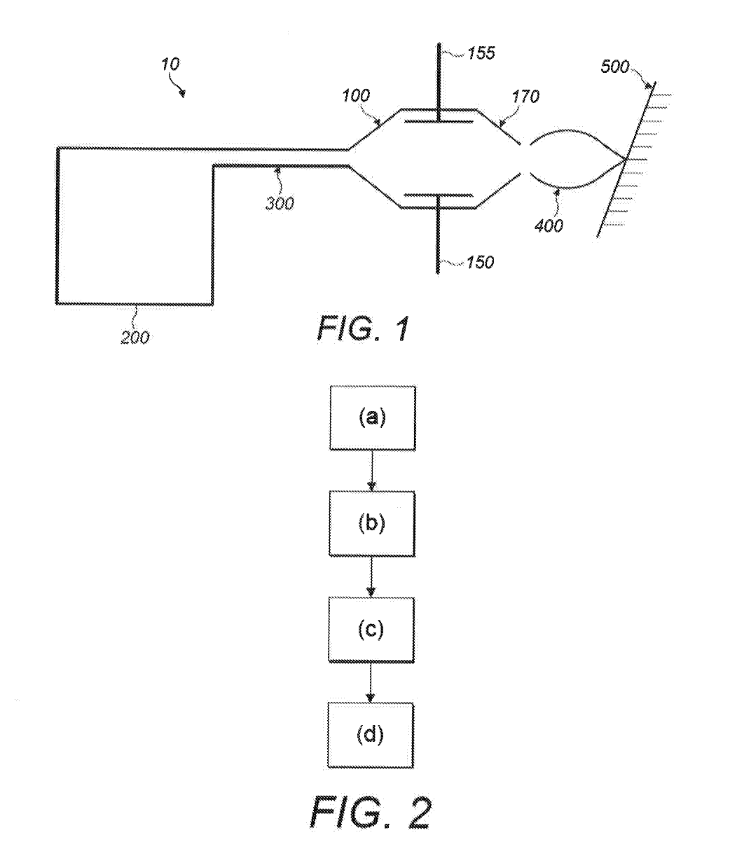

FIG. 1 shows a schematic representation of a plasma device;

FIG. 2 shows a flow chart if the key process steps;

FIG. 3 shows rehydration kinetics after 1 minute of plasma treatment on filed and unfilled nails; and

FIG. 4 shows rehydration kinetics after 1, 5 and 15 minutes of plasma treatment on unfilled nails.

in FIGS. 3 and 4, the key corresponds as follows:

A—Make up of lost water (Ar/He—PF4v2) 1 Min

A′—2 per. Mov. Avg. (Make up of lost water (Ar/He—PF4v2) 1 Min)

B—Make up of lost water (Ar/He—PF4v2) 1 Min filed

B′—2 per. Mov. Avg. (Make up of lost water (Ar/He—PF4v2) 1 Min filed)

C—Make up of lost water (Ar/He—PF4v2) 1 Min

C′—2 per. Mov. Avg. (Make up of lost water (Ar/He—PF4v2) 1 Min)

D—Make up of lost water (Ar/He—PF4v2) 5 Min

D′—2 per. Mov. Avg. (Make up of lost water (Ar/He—PF4v2) 5 Min)

E—Make up of lost water (Ar/He—PF4v2) 15 Min

E′—2 per. Mov. Avg. (Make up of lost water (Ar/He—PF4v2) 15 Min).

FIG. 1 shows a plasma device 10.

The plasma device 10 comprises: a source of gas 200 in communication with a plasma nozzle assembly 100 via a gas conduit 300. The plasma nozzle assembly 100 comprises first and second electrodes 150, 155 arranged such that gas can flow between them and such that a plasma may be formed by an electrical discharge between them.

The plasma nozzle assembly 100 further comprises a nozzle 170, downstream of the first and second electrodes 150, 155. The nozzle 170 is arranged such that a flow of plasma from the first and second electrodes 150, 155 forms a jet 400, which can be directed at a target 500.

In use, gas flows from source of gas 200 via gas conduit 300 to the plasma nozzle assembly 100. A voltage is applied across first and second electrodes 150, 155 to generate an electric field extending therebetween. The gas passes between the first and second electrodes 150, 155 to form a plasma, which exits nozzle assembly via nozzle 170 as a jet 400.

In FIG. 2, the steps (a)-(d) are shown. These steps involve: (a) applying the plasma to the infected nail or skin; (b) rehydrating the infected nail or skin; (c) applying the plasma to the infected nail or skin; and (d) optionally rehydrating the infected nail or skin.

The rehydration is achieved with water or a water-containing composition and will, typically substantially comprise water for rehydrating the nail. The water may, optionally, further comprise active ingredients for further treating the nail or skin surface. These active ingredients include, for example, hydrogen peroxide for additionally bleaching a surface or amounts of acid or base or salts to buffer active compounds which are formed by the plasma treatment.

In a further embodiment, regions of the nail or skin adjacent the treatment area may be masked off or protected from the application of plasma. This ensures that unnecessary tissue damage is avoided.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. Non-thermal plasma for use in the treatment of an infected nail or infected skin, wherein the plasma is used in a method comprising:
(a) applying the plasma to the infected nail or skin;
(b) rehydrating the infected nail or skin;
(c) applying the plasma to the infected nail or skin; and
(d) optionally rehydrating the infected nail or skin.
wherein the plasma is applied to a portion of the nail or skin in each of steps (a) and (c) until a hydration level of the plasma-treated portion drops by at most 30 wt % based on the initial moisture content of the plasma-treated portion, preferably by at most 20 wt %.

2. The plasma for use according to claim 1, wherein the plasma is applied to the nail or skin in each of steps (a) and (c) until a hydration level of a plasma-treated portion drops by at most 10 wt % based on the initial moisture content of the nail or skin.

3. The plasma for use according to claim 1, wherein rehydrating the nail or skin in each of steps (b) and (d) restores a hydration level of the nail or skin substantially to the initial moisture content of the untreated nail or skin.

4. The plasma for use according to claim 1, wherein:
(i) the steps (a)-(d) do not overlap; and/or
(ii) the method comprises repeating steps (b) and (c) at least once, preferably at least 3 times.

5. The plasma for use according to claim 1, wherein the nail or skin is infected with a viral, bacterial or fungal infection.

6. The plasma for use according to claim 5, wherein the nail or skin is infected with Trichophyton rubrum, or the skin is infected with a human papilloma virus.

7. The plasma for use according to claim 5, for treating warts, athlete's foot and/or Onychomycosis.

8. The plasma for use according to claim 1, wherein the plasma is provided by an electrical discharge through a gas, preferably through a gas selected from Helium, Oxygen, Argon, Krypton, Neon, Air, Hydrogen, and mixtures of two or more thereof.

9. The plasma for use according to claim 1, wherein in each of steps (a) and c) the plasma is applied to the portion of the nail or skin to be treated for a period of from 5 seconds to 2 minutes, preferably 15 seconds to 1 minute.

10. The plasma for use according to claim 1, wherein the skin or nail is rehydrated in each of steps (b) and (d) by applying water to a surface of the skin or nail and then removing excess water from the surface of the skin or nail.

11. The plasma for use according to claim 10, wherein water is applied to the skin or nail for a period of from 15 seconds to 6 minutes, preferably from 1 minute to 3 minutes.

12. The plasma for use according to claim 1, wherein the method comprises
(A) applying the plasma to the nail for a period of about 30 seconds;
(B) rehydrating the nail by applying water to the nail for a period of about 2 minutes; and
(C) repeating steps (A) and (B) at least three times.

13. The plasma for use according to claim 1, wherein, before step (a), the infected nail or skin is filed or abraded.

14. A non-therapeutic method for lightening the colour of a nail, wherein the method comprises:
(a) applying non-thermal plasma to at least a portion of the nail;
(b) rehydrating said portion the nail;
(c) applying non-thermal plasma to said portion of the nail; and
(d) optionally rehydrating said portion of the nail.

15. Non-thermal plasma for use in the treatment of an infected nail or infected skin, wherein the plasma is used in a method comprising:
(a) applying the plasma to the infected nail or skin;
(b) rehydrating the infected nail or skin;
(c) applying the plasma to the infected nail or skin; and
(d) optionally rehydrating the infected nail or skin
wherein the skin or nail is rehydrated in each of steps (b) and (d) by applying water or a water-containing composition to the skin or nail and wherein, before step (c), excess water is removed from the skin or nail.
